(19) United States
(12) Patent Application Publication
(10) Pub. No.: US 2018/0271690 A1
(43) Pub. Date: Sep. 27, 2018

(54) BOOSTER DEVICE FOR MUSCLES

(71) Applicant: ELIEZER EDELSTEIN, HAIFA (IL)

(72) Inventor: ELIEZER EDELSTEIN, HAIFA (IL)

(21) Appl. No.: 15/767,663

(22) PCT Filed: Oct. 20, 2016
(86) PCT No.: PCT/IL2016/051135

§ 371 Date: Apr. 12, 2018

Related U.S. Application Data
(60) Provisional application No. 62/244,215, filed on Oct. 21, 2015.

(51) Int. Cl. A61F 5/01; A61H 3/00

(52) U.S. Cl. CPC A61F 5/0125; A61H 3/00; A61H 2201/1676; A61F 2005/0179; A61H 2201/165; A61F 2005/0158

(57) ABSTRACT

A device for aiding a human joint, the device including a booster mechanism including a two part housing, a first part of the housing having a first end adapted for coupling adjacent one side of the joint and a second part of the housing having a second end adapted for coupling adjacent a second side of the joint, one part of the housing being movable relative to the other part of the housing, a spring mounted between the first part of the housing and the second part of the housing, a locking mechanism in the housing for releasably locking the spring when the spring is in a loaded state, and a release element engaging the locking mechanism for unlocking and releasing the loaded spring to boost the first end of the housing away from the second end of the housing.

### RELATED APPLICATIONS

This application claims the benefit of U.S. provisional patent application No. 62/244,215, filed 21 Oct. 2015.

### FIELD OF THE INVENTION

The present invention relates to booster devices for muscles, in general and, in particular, to a knee brace with a mechanism to aid a person to stand up.

### BACKGROUND OF THE INVENTION

There are many reasons that the muscles or joints of various people become weakened—disease, age, injury, etc. When, due to weakened joints and muscles such as the knees and skeleton, it becomes difficult to stand up and to sit down unaided or without pain, a support device is required. A number of solutions have been proposed for this problem which provide mechanical support particularly to the knee joints to aid a person when standing up and sitting down.

### SUMMARY OF THE INVENTION

The present invention relates to a booster device for enhancing the action of weakened muscles or joints, particularly the knees. Thus, there is provided, according to the invention, a device for aiding a human joint, the device including a booster mechanism including a two part housing, a first part of the housing having a first end adapted for coupling adjacent one side of the user's joint and a second part of the housing having a second end adapted for coupling adjacent a second side of the user's joint, and a swing initially loaded and disposed between the first part of the housing and the second part of the housing. The device further includes a plunger having a stopper for neutralizing the boosting operation of the spring in the initially loaded state. One part of the housing selectively moves relative to the other. When bending the joint, the first part of the housing approaches the second part of the housing thereby loading the spring further thus causing the spring to contain additional energy to be selectively released. The device also includes a locking mechanism in the housing, for releasably locking the spring containing the additional energy thus neutralizing the boosting operation of the spring in that state, and a release element in the housing engaging the locking mechanism for selectively unlocking and releasing the additional energy of the spring to boost the first part of the housing away from the second part of the housing

### BRIEF DESCRIPTION OF THE DRAWINGS

The present invention will be further understood and appreciated from the following detailed description taken in conjunction with the drawings in which:

FIG. 1a is an illustration of a device and method for boosting human joint muscles, constructed and operative in accordance with some embodiments of the present invention;

FIG. 1b is a blown illustration of a device and method for boosting human joint muscles, as illustrated in FIG. 1a constructed and operative in accordance with some embodiments of the present invention;

FIGS. 2a, 2b and 2c are illustrations of the operation in various states of the device and method for boosting human joint muscles, as illustrated in FIGS. 1a and 1b, according to some embodiments of the invention;

FIGS. 3a and 3b are illustrations of the operation in other states of the device and method for boosting human joint muscles, as illustrated in FIGS. 1a and 1b, according to some embodiments of the invention;

FIG. 4 is illustrations of various states of use of the device and method for boosting human joint muscles by a user, as illustrated in FIGS. 1a and 1b, according to some embodiments of the invention;

FIG. 5 is an illustration of the device and method for boosting human joint muscles, constructed and operative as shown in FIGS. 1a and 1b in accordance with some embodiments of the present invention;

FIG. 6a is an illustration of the device and method for boosting human joint muscles, constructed and operative in accordance with some embodiments of the present invention;

FIG. 6b is illustration of use of the device and method for boosting human joint muscles by a user, as illustrated in FIGS. 6a, according to sonic embodiments of the invention;

FIG. 7a is an illustration of a device and method for boosting human joint muscles, constructed and operative in accordance with some embodiments of the present invention;

FIG. 7b is a blown illustration of a device and method for boosting human joint muscles, as illustrated in FIG. 7a constructed and operative in accordance with some embodiments of the present invention;

FIGS. 8a, 8b, 8c, 8d, 8e and 8f are illustrations of the operation in various states of the device and method for boosting human joint muscles, as illustrated in FIGS. 7a and 7b, according to some embodiments of the invention;

### DETAILED DESCRIPTION OF THE INVENTION

The present invention relates to a booster device to add power to weak or weakened muscles or joints. The device is particularly useful for enhancing the action of weakened leg muscles and is described herein with relation to this use. However, it will be appreciated that the device can be adapted for boosting weak muscles of other joints. The device is useful for providing additional power to the knee to lift the body when a person stands up and adding support to the knee muscles when a person sits down. Consequently, it reduces the load on the knees.

The booster device includes a booster mechanism having a two part housing, where the first part and the second part can move relative to one another. The first part of the housing has a first end adapted and configured for coupling adjacent one side of the joint and the second part of the housing has a second end adapted and configured for coupling adjacent a second side of the joint. The booster mechanism further includes an initially loaded spring mounted in the device by disposing it between the first and second parts of the housing and a plunger having a stopper adapted and configured to neutralize the boosting operation of the spring in the initially loaded state so as to allow free movement of the first part of the housing relative to the second part of the housing.

When the first end of the first part of the housing selectively approaches the second end of the second part of the housing, while bending the joint, additional energy accumulates in the initially loaded spring which, when selectively released, pushes the first end away from the second end, to straighten the joint. The booster mechanism further includes a locking mechanism for releasably locking the spring containing the additional energy in the loaded state so as to allow free movement of the first part of the housing relative to the second part of the housing. A release element is also provided in the housing, engaging the locking mechanism, for selectively unlocking the spring and releasing the additional energy of the spring to boost the movement of the first part of the housing away from the second part of the housing.

The booster device according to an embodiment of the invention, may include or may be connected to a knee brace mountable to the legs. One part of the booster is attached to the thigh and another part of the booster is attached to the shin, as by straps or other attaching element. The booster device is adapted and configured to load a spring with additional energy when the joint pivots from a straight orientation to a bent orientation of the knee so that the loaded spring contains power to assist and boost the wearer's muscles and joints to pivot from a bent orientation to a straight orientation of the knee, when the additional energy of the spring is released.

Preferably, the operating range of the booster's device or mechanism is divided into three zones: a) around the straight knees position, while the user is standing or walking, wherein the initially loaded spring is neutralized by the plunger stopper so as to allow the two parts of the housing to move freely relative to one another to permit unencumbered walking; b) while the user is in a sitting position, around the fully bent position of the knees, wherein the spring is loaded and contains additional energy, the locking mechanism locks the spring thus neutralizing its boosting operation which allows the two parts of the housing to move freely relative to one another and permits free movement of the knees in the sitting position; and c) when the user is in the process of sitting down or getting up. When the user is in the process of sitting down, the initially loaded spring is loaded with additional energy by absorbing the force exerted by the weight of the user acting on the user's knees, and is locked and neutralized as described in zone b) above. While the user is in the process of standing up, the spring is released to its original initially loaded state and the booster mechanism is operative to add power to the knee muscles to straighten the knee.

The booster mechanism can include a mechanical spring, a spring piston, an hydraulic piston, or any other suitable mechanism that can be loaded and contain potential energy while supporting the weight of the user's body when a user bends his or her knees while in the process of sitting down, and that uses the stored potential energy to pivot and straighten or unfold the booster device when unlocked, to push a person from a sitting position to a standing position.

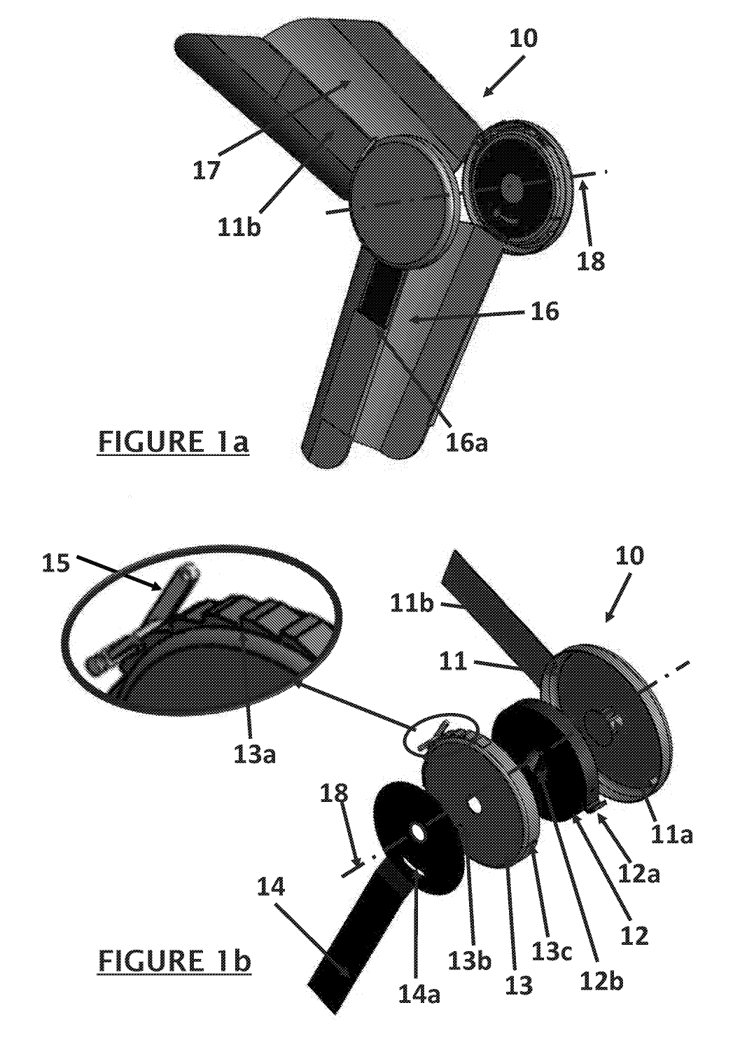

Referring now to FIGS. 1a and 1b, there is shown an example of a booster mechanism 10 utilizing a spring force created by rotation and fulfilling all of the system requirements, according to embodiments of the invention. It is described, by way of non-limiting example only, with regard to providing power to a user's knees. The booster mechanism 10 includes a two part housing—an outer part 11 and an inner part 13. An initially loaded torsion spring 12, having a first bent end 12a and a second bent end 12b, is mounted between the outer part 11 and the inner part 13. An arm 14 is coupled to inner part 13. A locking mechanism associated with a release element, here illustrated as a locking arm 15 and ratchet 13a, is coupled to inner part 13, as described in detail below.

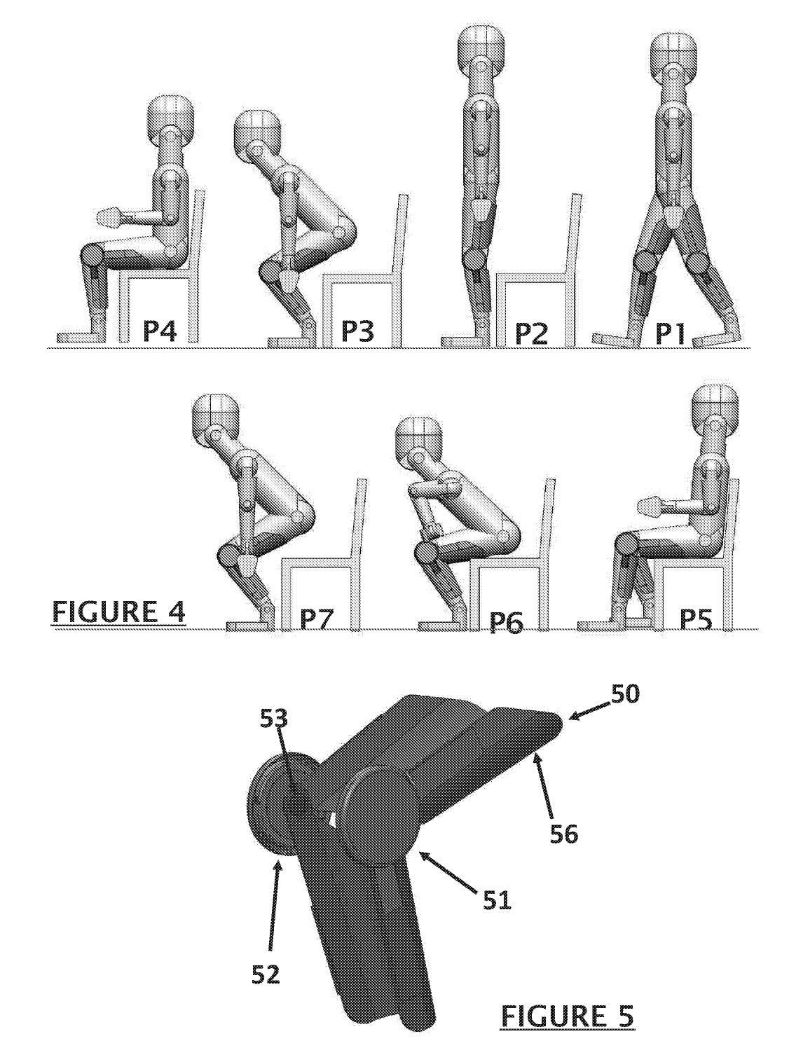

Booster mechanism 10 is shown in FIG. 1a in an exemplary brace including a thigh brace part 17 and a shin brace part 16. While wearing the brace with the booster mechanism, the thigh brace part 17 is attached to the thigh of the user's leg and shin brace part 16 is attached to the shin of the leg, as by strips or straps (not shown). The thigh brace part 17 and the outer part 11 are fixedly connected tightly together. The arm 14 slides in slots 16a of the shin brace part 16 of the brace. The arm 11b is affixed to the thigh brace part 17 of the brace. The inner part 13, the arm 14 and the outer part 11 pivot around a common axis 18. The spring 12 operates between the outer part 11 and the inner part 13. Bent end 12b is held by a slot in axle 11a on the outer part 11, and bent end 12a is held by slot 13c in the inner part 13. While sitting down (as shown in FIG. 4, position P3), parts 16 and 17 rotate relative to one another, causing the inner part 13 to rotate relative to the outer part 11. Thus, torque is developed between the two parts, loading or unloading the spring. A locking mechanism, here implemented as a toothed arc or ratchet 13a with a locking arm 15, is attached to, or formed with the inner part 13. In addition, a pin 13b extends from part 13 and slides in an arched slot 14a formed in part 14. While starting to sit down, the knees bend, shin brace part 16 together with arm 14 are rotated relative to the thigh brace part 17 together with outer part 11. After rotation through a pre-selected angle, the side edge of slot 14a engages pin 13b, forcing the inner part 13 to further rotate together with part 14, and thereby further loading the torsion spring 12. Thus, additional torque energy is developed between the outer part 11, attached to the thigh brace part 17, and arm 14, attached to the shin brace part 16 reducing the load off the knee, while the spring 12 supports a portion of the body weight of the user.

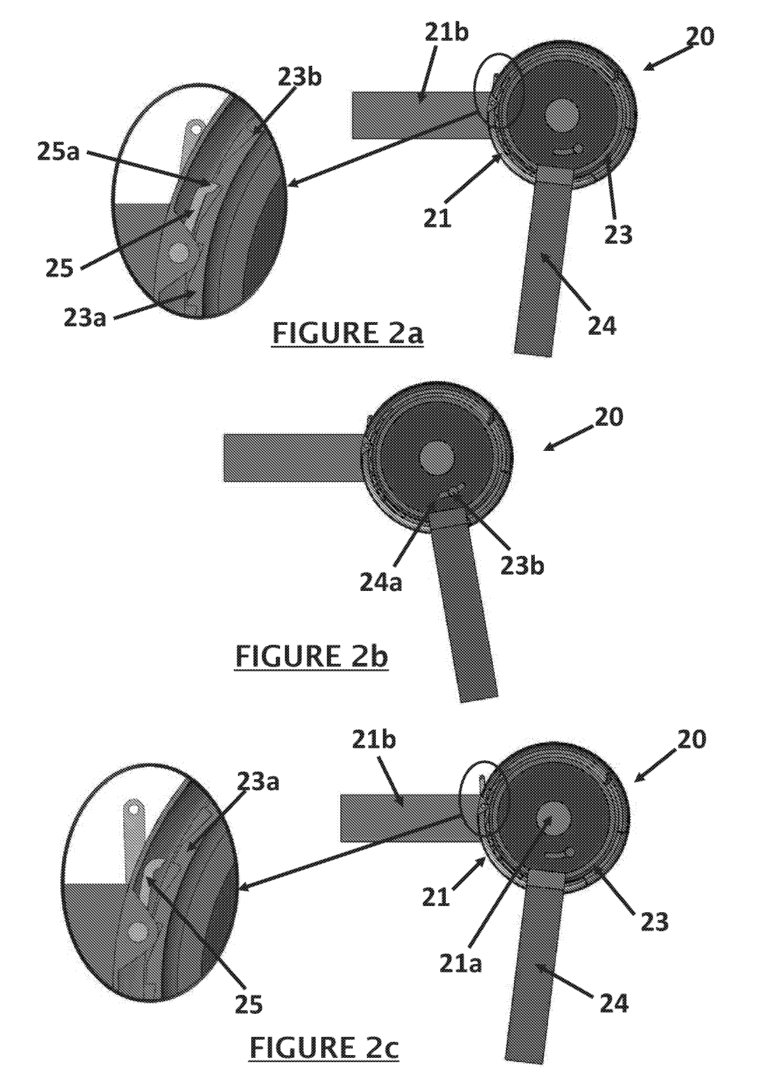

FIGS. 2a, 2b and 2c, show an exemplary booster mechanism when the user is in a sitting position. As the thigh brace part rotates through a pre-defined angle relative to the shin brace part, a tooth 25a of locking arm 25 falls into a slot 23b in the toothed arc 23a. As the knee bends more, tooth 25a of the locking arm 25 falls into the next slot in the toothed arc 23a, until the person reaches a comfortable sitting position (as shown in FIG. 4, position P4). At that stage, the inner part 23 is locked relative to the outer part 21 by the locking mechanism (arm 25 and toothed arc 23a), the engagement of toothed arc 23a by the locking arm 25a, neutralizes the boosting operation of spring 12 in the booster mechanism 20, i.e., no additional torque is transferred from the brace to the spring 12 and no torque is imparted from the spring to the brace. Arm 24 is free to move through a limited arc relative to the inner part 23, due to slot 24a, so the person can freely move his knees (as shown in FIG. 4, position P5).

In order to stand up, the knees are bent and the weight of the person further loads the brace and the booster mechanism spring (as shown in FIG. 4, position P6). The spring (not shown) mounted about axle 21a of the outer part 21 is further loaded while arm 21b rotates relative to the inner part 23. The user rotates locking arm 25, now acting as a release mechanism, in order to unlock the booster mechanism, thus lifting tooth 25a from the slots in the toothed arc 23a and releasing the spring, which boosts or adds power to the person's knee muscles while getting up (as shown in FIG. 4, position P7).

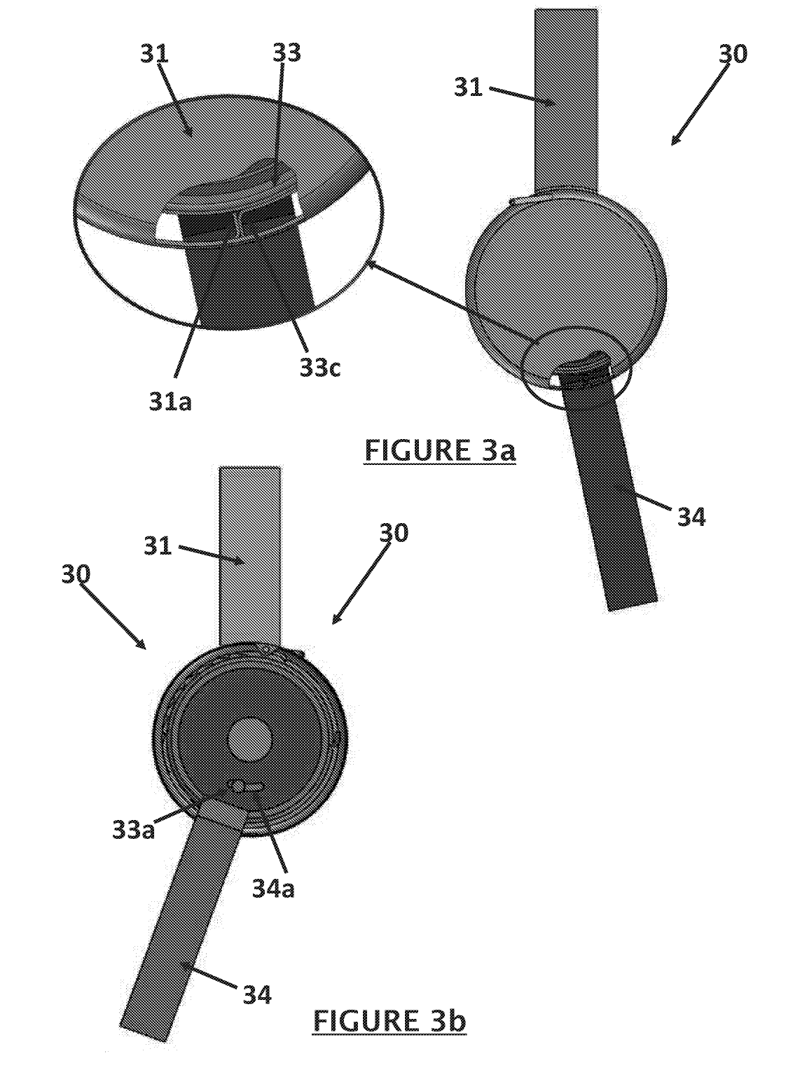

As shown in FIGS. 3a and 3b, when the user is close to a standing position, i.e., when the knee and the booster mechanism form a standing leg angle_(as shown in FIG. 4, position P2), stopper 31a of the outer part 31 engages stopper 33c of the inner part 33. This neutralizes the boosting operation of the spring, as the spring is locked in place between the inner part 33 and the outer part 31 in a loaded state, and the spring force is not transferred to the arm 34 or to the brace. Thus, arm 34 is free to move through a limited arc relative to the inner part 33 and outer part 31, as pin 33a slides along the arc of slot 34a, as shown in FIG. 3b. In this way, the user can walk freely, as shown in FIG. 4, position P1.

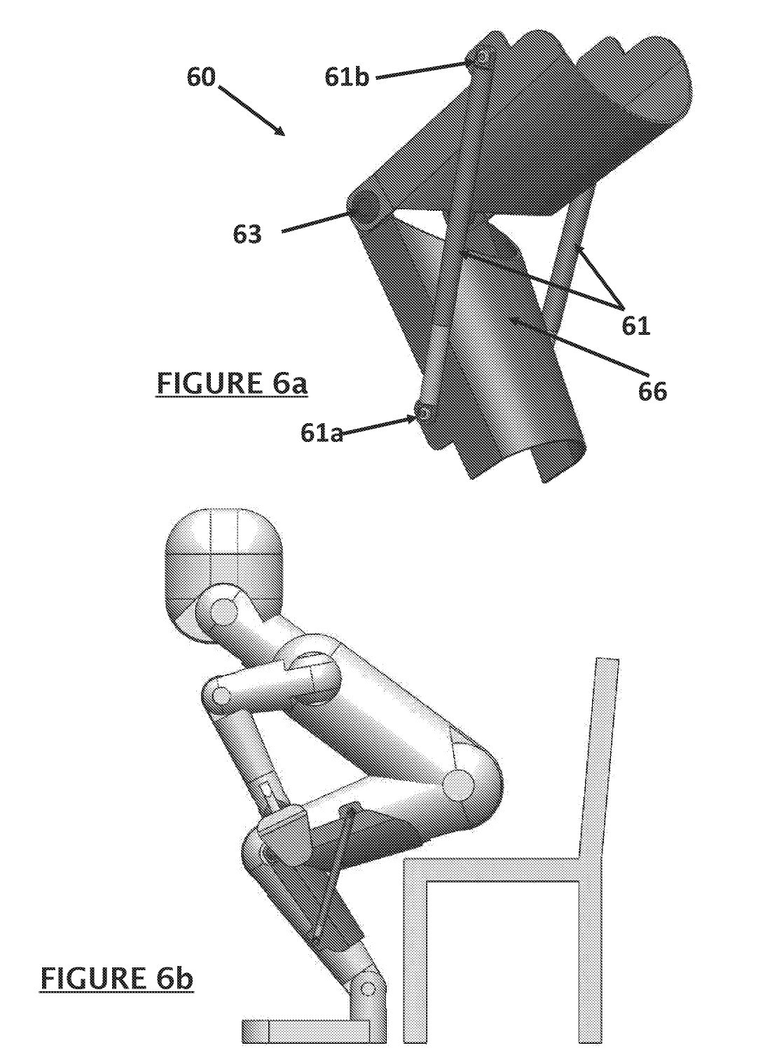

According to another embodiment of the invention, as shown in FIG. 5, a left booster mechanism 51 and a right booster mechanism 52 can be attached to a knee brace 56 that has a pair of hinges 53 of its own. The axles of all the hinges, of the knee brace and of the booster mechanisms, are mounted concentrically. It will be appreciated that all of the booster mechanisms described herein are operative in this embodiment, as well.

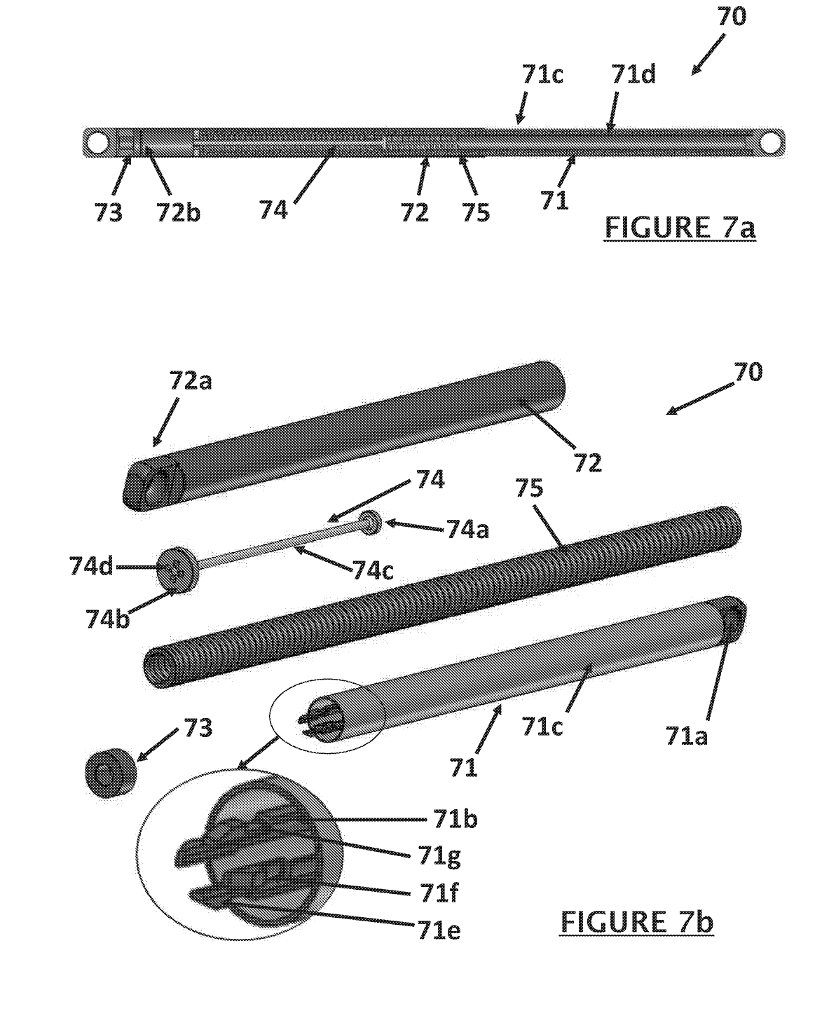

According to another embodiment of the invention, the booster mechanism can utilize a spring force created by linear motion, as in a piston booster mechanism, as shown in FIGS. 6a and 6b. In this embodiment, two piston boosters 61 are attached to a knee brace 66, fulfilling all of the system requirements. It will be appreciated that this embodiment can be used as a stand-alone device, by strapping to a user's thigh and shin or can be affixed, on concentric hinge axles, to an existing knee brace.

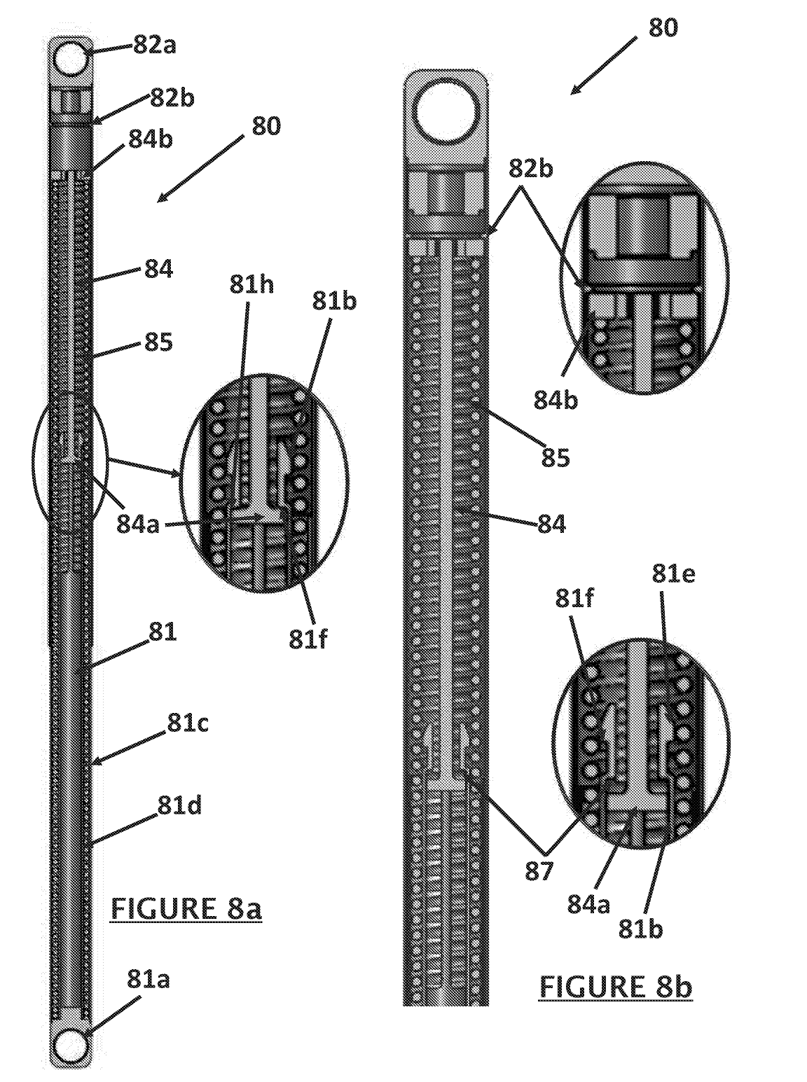

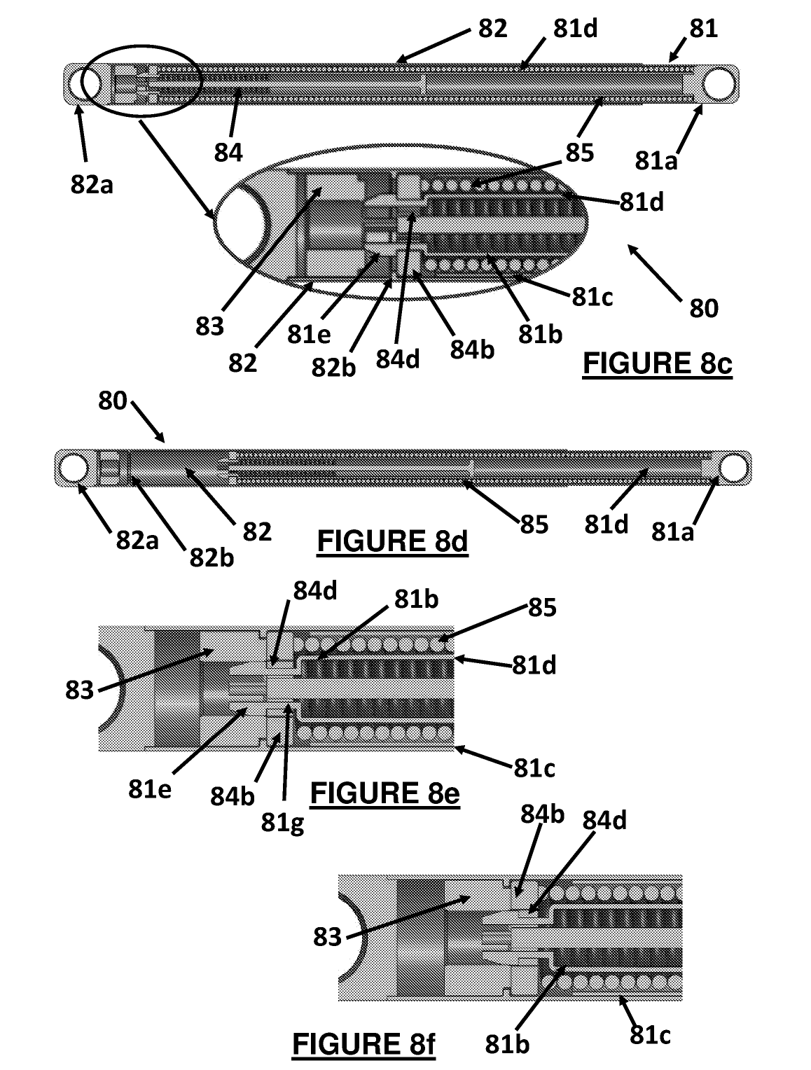

FIGS. 7a and 7b are assembled and exploded views, respectively, of one example of a piston booster 70, suitable for use in the booster mechanism shown in FIG. 6a. Piston booster 70 includes a two part housing, here a hollow, double-walled inner cylinder 71, slideably disposed inside a hollow outer cylinder 72. The inner cylinder 71 includes an outer wall 71c and an inner wall 71d. A pivot mount 71a is mounted on one end of inner cylinder 71. Pivot mount 71a can be integrally formed with inner wall 71d and outer wall 71c, as part the inner cylinder 71. The other end of inner wall 71d includes a locking mechanism, here implemented as at least two prongs 71b, better seen in FIG. 8b as prongs 81b, which are extensions of the inner wall 71d of the inner cylinder 71. The end portion of prong 71b, in the illustrated embodiment, includes a shoulder 71f, a neck portion 71g having a narrower diameter than the inner wall 71d of the inner cylinder 71, and a tapered anchor 71e. A pivot mount 72a is attached to one end of the outer cylinder 72. A release mechanism, here implemented as an unlocking ring 73, is disposed inside outer cylinder 72 between pivot mount 72a and a stopper ring 74b. The other end of outer cylinder 72 is open and receives the inner cylinder and the spring.

Piston booster 70 also includes a plunger 74. Plunger 74 includes a plunger rod 74c with a plunger stopper 74a on one end and an anchoring disk 74b on the other end. Anchoring disk 74b includes at least one, and preferably a plurality of apertures 74d, in registration with the prongs 71b and adapted and configured to permit the passage of tapered anchors 71e and to engage neck portions 71g of the prongs 71b, as described in detail below, which serve as a releasable locking mechanism for the booster device by locking the spring and neutralizing its boosting operation. While it is preferred that there by one aperture for each prong, it is possible to utilize a single arcuate aperture which can accommodate several prongs. Plunger stopper 74a is slidingly disposed between prongs 71b, and the plunger rod 74c with the anchoring disk 74b extends into outer cylinder 72 beyond anchors 71e.

Plunger stopper 74a has a larger diameter than neck portion 71g, so it is slideably locked between prongs 71b. A spring 75 is disposed between the inner wall 71d and the outer wall 71c of inner cylinder 71 and engages pivot mount 71a at one end, and anchoring disk 74b of the plunger at its other end. Spring 75 is mounted initially loaded and locked between anchoring disk 74b and pivot mount 71a.82b.

FIGS. 8a-8f show the configurations of piston 80 in operation, for various positions of the user, including sitting, walking, getting up and sitting down.

FIG. 8a shows the piston booster fully extended and disengaged, for ease walking of the user. The spring 85 is blocked between anchoring disk 84b and the inner cylinder 81 at the pivot mount 81a. The plunger stopper 84a abuts shoulder 81f of prongs 81b due to its larger diameter than the inner cylinder at neck portions 81g of the prongs. While walking, the boosting operation of the spring in the piston is neutralized. At a pre-selected point during motion of the inner cylinder outwards from the outer cylinder, the plunger stopper 84a abuts against shoulders 81f of the prongs 81b, as shown in FIG. 8a. Since the spring 85 is blocked the outer cylinder 82 can slide freely relatively to the inner cylinder 81 and the distance between the outer cylinder pivot 82a and the inner cylinder pivot 81a can change freely to enable the user to freely move his legs.

As shown in FIG. 8b, while the user is in the process of sitting down, the piston booster 80 gets shorter, i.e., the inner cylinder 81 slides into the outer cylinder 82. At a pre-selected point during motion of the inner cylinder into the outer cylinder, the anchoring disk 84b on the end of plunger 84 abuts against the stopper ring 82b disposed in the outer cylinder 82. As the inner cylinder continues to slide into the outer cylinder, the plunger stopper 84a disengages from shoulders 81f of the prongs 81b, creating a gap 87 and further loading the spring. Continued sliding of the inner cylinder into the outer cylinder 82 further loads the spring 85 by compressing the spring between the anchoring disk 84b and the pivot mount 81a. From that point on, as the piston continues to contract, the load on spring 85 continues to grow.

Referring now to FIGS. 8c and 8d, there is shown the piston booster at bent and constricted orientations of the booster mechanism while a user is sitting down. As the user reaches a sitting position, the piston booster will lock the spring in its additionally loaded state. In this way, the boosting power of the spring is neutralized, so the piston's pivot mounts 81a and 82a can pivot freely relative to the brace (and the user's knees), to let his feet move freely during sitting. Locking of the spring is achieved by anchoring the prongs 81b of the inner cylinder 81 to the anchoring disk 84b of the plunger 84, as shown in FIG. 8c. In this configuration, where the anchoring disk 84b is anchored to the inner cylinder 81, the spring is locked in its most compressed orientation between the anchoring disk 84b and the pivot mount 81a. This permits the outer cylinder 82 to move freely relative to the inner cylinder 81, letting the person move his or her knees freely while sitting. This range of motion is exemplified in FIGS. 8c and 8d.

In the illustrated embodiment, locking of the spring is implemented by passage of anchors 81e of prongs 81b through apertures 84d in anchoring disk 84b until the disk is engaged by necks 81g. It will be appreciated that the ends of anchors 81e are tapered for ease of entry of the anchor into apertures 84d of the anchoring disk 84b. As they pass through the apertures 84d, the tapered shape causes the flexible prongs 81b to bend and create an elastic force so that when the anchors 81e pass through the apertures 84d and the neck portion is in the aperture, the prongs 81b spring back so as to lock the prongs in the anchoring disk 84b with the neck portions of the prongs engaging the disk 84b. In this state, the anchors 81e lockingly engage the disk 84b so that the anchors 81b cannot be pulled back through the apertures 84d of disk 84b, thereby locking the spring in its loaded state and neutralizing the boosting action of the spring. In this position, the inner cylinder 81 is free to slide in and out of the outer cylinder 82 while the spring 85 remains loaded and locked, allowing the user to bend and straighten his knees freely, while sitting.

When the user is ready to stand, the spring is released by releasing the anchors 81e of the inner cylinder 81 from the anchoring disk 84b of the plunger 84. As the spring expands, the piston extends, providing a boost to the user's knees to support the user while standing up and to reduce the load off the knees and its muscles. This process is shown, in the illustrated embodiment, in FIGS. 8e and 8f.

FIG. 8e shows the unlocking of the anchors 81e from the anchoring disc 84b, according to the illustrated embodiment of the present invention. To start getting up, the user brings his legs to a getting up position, moves his body weight forward, from the chair to the brace, thereby applying force to the piston booster (i.e., pushing the inner cylinder further into the outer cylinder) and compressing the spring a bit more, causing the tapered anchors 81e to slide into the central bore of the unlocking ring 83. At this stage, unlocking ring 83 squeezes together the tapered anchors 81e. The bore of the unlocking ring 83 has a circumference similar to the circumference of a circle drawn about the outer edges of all the locking apertures 84d in the anchoring disk 84b. Once the anchors are squeezed towards one another into the bore of unlocking ring 83, the neck portions 81g of prongs 81b release the anchoring disk 84b and the anchors can easily slip out through the apertures 84d in the anchoring disk 84b, thereby releasing the prongs 81b. FIG. 8f shows the anchors 81e passing through the anchoring disk 84b. Once the prongs are released, the power of the loaded spring causes rapid expansion of the spring which, in turn, causes the inner cylinder to slide out of the outer cylinder, thus boosting the action of the user's knees and helping him stand up.

It will be appreciated that the booster mechanism is configured so that only when the user places his or her weight on the booster when starting to stand will there be sufficient force to drive the anchors to slide along their tapered ends into the central bore of the unlocking ring 83 and release the spring. Preferably, the weight or force required to release the booster mechanism is adjustable, for example, according to the weight of the user.

It will be appreciated that the booster mechanisms described in detailed hereinabove are only exemplary illustrations of the system, for explanation and comprehension purposes. Alternatively, the loading of the brace can be implemented by any other spring force device, such as a buckling spring, a magnetic spring, etc.

While the invention has been described with respect to a limited number of embodiments, it will be appreciated that many variations, modifications and other applications of the invention may be made. It will further be appreciated that the invention is not limited to what has been described hereinabove merely by way of example. Rather, the invention is limited solely by the claims which follow.

1. A device for aiding a human joint, the device comprising:
a booster mechanism including a two part housing, a first part of the housing having a first end adapted for coupling adjacent one side of the joint and a second part of the housing having a second end adapted for coupling adjacent a second side of the joint, one part of the housing being movable relative to the other part of the housing;
a spring mounted between the first part of the housing and the second part of the housing;
a locking mechanism in the housing for releasably locking the spring when the spring is in a loaded state; and
a release element engaging the locking mechanism for unlocking and releasing the loaded spring to boost the first end of the housing away from the second end of the housing.

2. The device according to claim 1, further comprising: a plunger having a plunger stopper for neutralizing the boosting operation of the spring in an initially loaded state.

3. The device according to claim 1, wherein:
the housing includes:
an outer part and an inner part coupled for relative rotational movement;
an arm pivotally coupled to the inner part; and
an arm coupled to the outer part;
the spring includes a torsion spring disposed between the outer part and the inner part;
and the locking mechanism includes a toothed arc and a locking arm, coupled to the inner part.

4. The device according to claim 1, wherein the booster mechanism is a piston booster;
the housing includes a double walled inner cylinder slideably mounted in an outer cylinder;
the spring is disposed between the walls of the inner cylinder and between the inner cylinder and the outer cylinder;
the locking mechanism includes:
at least two prong extensions of the inner wall, each prong defining a shoulder, a neck portion and a tapered anchor;
a plunger having a plunger rod with a plunger stopper at one end and an anchoring disk at the other end, the anchoring disk including a plurality of apertures in registration with the prongs; and
the release element includes an unlocking ring disposed in the second part of housing adjacent the locking mechanism on the first part.

5. The device according to claim 1, further comprising a brace including a thigh brace part and a shin brace part; the first end of the first housing engaging the thigh brace part and the second end of the second housing engaging the shin brace part.

6. The device according to claim 5, wherein the joint to be aided is a knee, and wherein the first end is adapated to be coupled to a thigh above the knee and the second end is adapted to be coupled to the shin below the knee.

7. The device according to claim 1, wherein the first and second parts can adopt three states:
a first state wherein the spring is initially loaded, locked and neutralized permitting free motion between the first and second parts of the housing;
a second state wherein the motion of the first and second parts of the housing towards one another further loads the spring, locks it and neutralizes its boosting power; and
a third state wherein the spring is released to its original state and the booster mechanism is operative to boost motion of the first and second parts of the housing away from one another.

8. A method for aiding straightening and bending of a human joint, the method comprising:
coupling a first end of a device having a booster mechanism on one side of the joint;
coupling a second end of the device to a second side of the joint;
wherein the booster mechanism includes
a two part housing, a first end of the device being on a first part of the housing and the second end being on a second part of the housing; and
a spring mounted between the first part of the housing and the second part of the housing; and
a locking mechanism in the housing for releasably locking the spring in a loaded state; and
a release element in the housing engaging the locking mechanism for unlocking and releasing the spring;
moving the first part of the housing relative to the second part of the housing to selectively load or release the spring.

9. The method according to claim 8, wherein the booster mechanism further includes a plunger having a plunger stopper for neutralizing the boosting operation of the spring in an initially loaded state.

10. The method according to claim 9, further comprising:
selectively moving the first end and the second end towards one another for loading the spring with boosting power;
providing resistance between the first end and the second end when the first end approaches the second end, when bending the joint.

11. The method according to claim 10, further comprising:
releasing the locked loaded spring when the joint is bent;
moving the first end and the second end away from one another upon release of the locked loaded spring; and
boosting the straightening of the joint.

12. The method according to claim 8, further comprising locking the spring and neutralizing the booster mechanism when the spring is loaded, for permitting free relative movement between the first end and the second end when the joint is bent.

13. The method according to claim 8, further comprising locking the spring and neutralizing the booster mechanism when the spring is initially loaded for permitting free relative movement between the first end and the second end when the joint is straight.
